(19) United States
(12) Plant Patent Application Publication
(10) Pub. No.: US 2009/0183287 P1
(43) Pub. Date: Jul. 16, 2009

(54) Raspberry named 'Nantahala'

(50) ;

(75) Inventors: James R. Ballington, Raleigh, NC (US); Gina E. Fernandez, Raleigh, NC (US); Susan K. Bryson, Fletcher, NC (US)

Correspondence Address: Gina E. Fernandez, Box 7609, 170 Kilgore Hall NCSY, Raleigh, NC, 27695, US

(73) Assignee: North Carolina State University, Raleigh, NC (US)

(21) Appl. No.: 11/998,754

(22) Filed: Jan. 15, 2008

(51) Int. Cl. A01H 5/00

(52) U.S. Cl. PLT/204

(57) ABSTRACT

Rubus idaeus Linnaeus ‘Nantahala’ is a new and distinct variety of raspberry that has the following unique combination of desirable features that are outstanding in a new variety.
1. Late season ripening to follow ‘Heritage’.
2. Fruit is medium size, 3.5 g.
3. Fruit is firm, very attractive, uniform and conical to ovate.
4. Consistent and moderate yields.

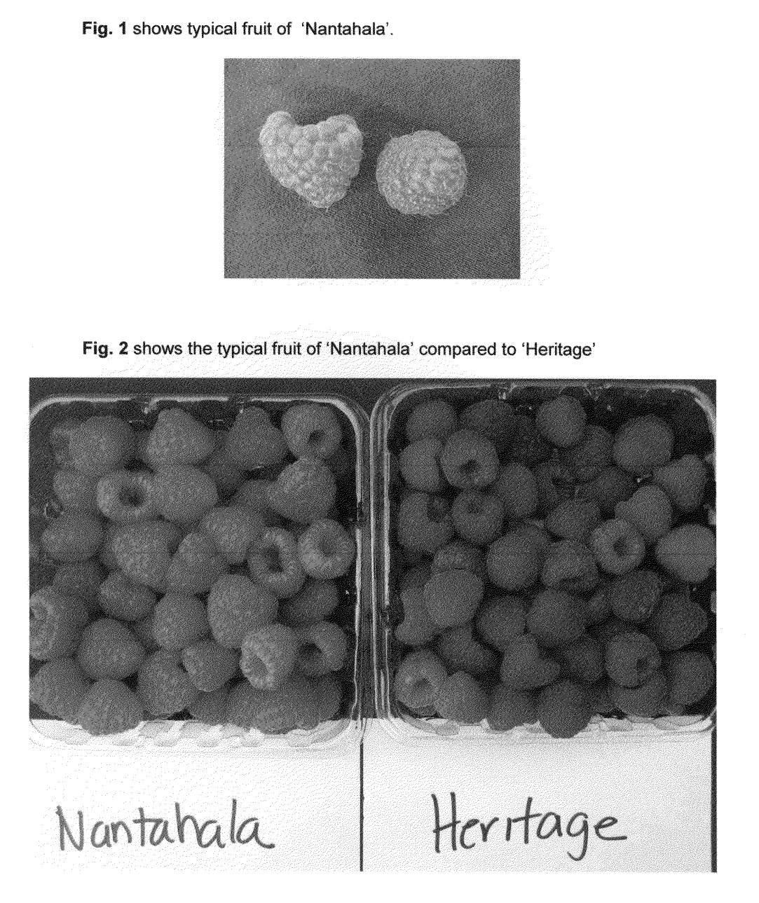

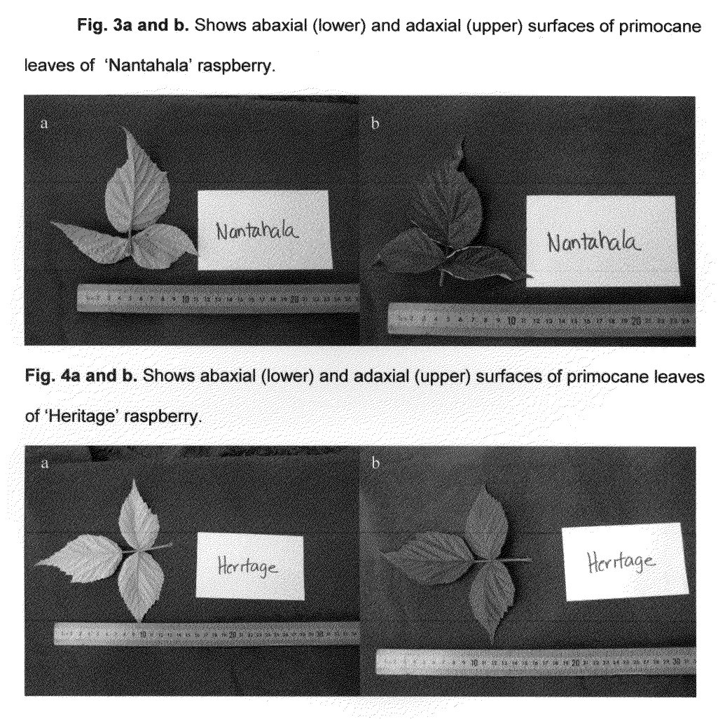

### LATIN NAME OF THE GENUS AND SPECIES

The Latin name of the novel raspberry variety disclosed herein is Rubus idaeus Linnaeus.

### VARIETY DENOMINATION

The inventive cultivar of Rubus idaeus disclosed herein has been given the variety denomination ‘Nantahala’.

### BACKGROUND OF THE INVENTION

The present invention related to a new and distinct cultivar of Rubus idaeus Linnaeus (raspberry) grown as a fruiting shrub for commercial agriculture. Raspberries are typically consumed both fresh and in a number of processed products.

The new and distinct variety of raspberry (Rubus idaeus Linnaeus) originated from the hand pollinated cross of NC 245 (unpatented)×Rossana (unpatented) made in 1994 in Raleigh, N.C. The seeds were germinated in the winter of 1994-1995 and the resulting seedlings were established at the Upper Piedmont Research Station in Reidsville, N.C. (GPS coordinates N36°, W0791') in the spring of 1995. When the seedlings had experienced 4 years of growth under field conditions in 1998, NC451 was selected for it large and firm berry and superior fruit flavor. The selection was then propagated by crown divisions and root cuttings. The propagules were planted in replicated trials with other raspberries at the Mountain Horticultural Research Station (GPS N35° W082') in Fletcher, N.C. and the Upper Mountain Research Station (GPS N36° W081', elevation 2846 ft.) in Laurel Springs, N.C. Plants and fruit of this new variety have remained true to type through successive cycles of asexual propagation. The new variety has been named the ‘Nantahala’ cultivar.

### SUMMARY OF THE INVENTION

‘Nantahala’ is a new and distinct variety of raspberry for fresh market production. ‘Nantahala’ berry is larger and firmer than ‘Heritage’ an industry standard. ‘Nantahala’ ripens later than most primocane fruiting cultivars and is recommended for the mountain regions of North Carolina and adjacent states with high elevation. In sensory evaluation panels, ‘Nantahala’ rated as good or better than ‘Caroline’, ‘Heritage’ and a store bought cultivar from California. In the Cherokee language, ‘Nantahala’ means “land of the midday sun.”

### BRIEF DESCRIPTION OF THE DRAWINGS

The photographs were made using digital photography techniques and illustrate the colors as true as reasonably possible when using these techniques. Colors in the photographs may differ slightly from the color values cited in the detailed botanical description, which accurately describe the colors of the new Rubus idaeus variety. All photographs were taken from plants grown at the Upper Mountain and Horticultural Crops Research Stations in Laurel Springs and Fletcher, N.C. respectively.

### DETAILED BOTANICAL DESCRIPTION OF THE VARIETY

The following is a detailed botanical description of a new and distinct variety of Rubus idaeus Linnaeus know as ‘Nantahala’. The observations below are from mature plants grown in test plots at a standard spacing of row width of 2 feet and 10 feet between rows. Those skilled in the art of cultivar description and evaluation will appreciate that certain characteristics of a variety will vary with older or conversely younger plants, as well as plants grown under different production protocols.

‘Nantahala’ has not been observed under all possible environmental conditions. Where dimensions, sizes, colors and other characteristics are given, it is to be understood that such characteristics are approximations or averages set forth as accurately as possible. The phenotype of the variety may differ from the description herein with variations in the environment such as season, temperature, light intensity, day length and cultural conditions. Color notations are based on The Royal Horticultural Society Colour Chart, The Royal Horticultural Society, London, UK, 1995 edition.

Yield components for ‘Heritage’, ‘Caroline’ (U.S. Plant Pat. No. 10,412) and ‘Nantahala’ can be found in Table 1. For botanical description purposes, ‘Nantahala’ was compared to the earlier ripening ‘Heritage’ a full description can be found in Table 2.

TABLE 1
Yield components of raspberries harvested from
Laurel Springs, and Fletcher, NC.

| | Laurel Springs, | | | |
|---|---|---|---|---|
| | NC Yield | Laurel Springs, | Fletcher, NC | Fletcher, |
| | estimate | NC Berry | Yield estimate | NC Berry |
| | lbs/acre¹ | Wt (g) | lbs/acre¹ | Wt (g) |
| Nantahala | 8107 | 3.5 | 9253 | 3.5 |
| Caroline | 12306 | 3.2 | 12583 | 2.5 |
| Heritage | NA | NA | 10178 | 2.9 |

¹Yields based on 3 years of replicated cultivar trials at each location, for a total of 6 years. Yield estimates were calculated using: Yield/cane of 3 canes/plot, * no. canes/plot. Yield estimation model from: Daubeny, H.A., A. Dale, , G. McGregor. 1986. Estimating yields of red raspberries in small research plots. HortScience: 2 1(5): 1216-1217.

The botanical descriptive data presented were collected from mature plants at the Upper Mountain Research Station in Laurel Springs, N.C. in 2006 and 2007.

Table 2 provides information on the plant and fruit characteristics of the new cultivar ‘Nantahala’. The new variety is particularly characterized and distinguished from other cultivars by its fruit firmness, superior sensory evaluation, and late harvest season.

TABLE 2
Plant and fruit characteristics of ‘Nantahala’ and ‘Heritage’.

| | ‘Nantahala’ | ‘Heritage’ |
|---|---|---|
| General | | |
| Plant size | Medium to large | Medium |
| growth habit | Erect | Erect |
| productivity | Low-Medium | Medium |
| self-fruitfulness | Self | Self |
| time of bud burst (Raleigh NC) | 1-Apr | 29-Mar |
| Primocane fruiting | | |
| percent of cane length | 25 | 30 |
| flowering as primocane | | |
| percent of total yield | 90 | 85 |
| No. fruiting nodes | 9-11 | 14-15 |
| Primocanes | | |
| number of young shoots/ft 2 | 12 | 8 |
| young shoot pigmentation | Medium | Medium |
| length (cm) | 151 | 126 |
| cane diameter at 15 cm | 8.1 | 7.6 |
| from ground (cm) | | |
| Cane diameter at 50 cm | 7.5 | 6.8 |
| from ground (cm) | | |
| Height: diameter at 15 | 19.1 | 16.6 |
| cm from ground | | |
| Height: Diameter at 50 | 20.9 | 18.8 |
| cm from ground | | |
| time of shoot emergence | 3-Apr | 31-Mar |
| Glaucosity (waxy bloom) | Weak | Weak |
| Cane Cross Section from | Round | Round |
| mid cane of primocane | | |
| Dormant cane color | 167C | 183B |
| Prickles | | |
| Pigmentation | Purple-brown | Red brown to brown |
| Density on young shoots | Moderate | Dense |
| Attitude of tip | Straight | Down |
| Texture | Smooth | Rigid |
| Presence and distribution | Present and | Present and |
| on petioles | Irregular | irregular |
| pubescence on canes | Absent | Absent |
| Internodal distance (cm) | 2.3 | 3.2 |
| at central ⅓ of cane | | |
| Density per 1 cm cane at 15 cm | 17 | 17 |
| from ground | | |
| Density per 1 cm cane at 50 cm | 5 | 6 |
| from ground | | |
| LEAVES | | |
| Color | | |
| Face | 137A | 137A |
| Relief between veins | Weak | Very weak |
| Glossiness | Medium | Medium |
| Underside | 148B | 148B |
| Petiole Length (cm) | 4.9 | 5.9 |
| Stipule orientation | Erect | Erect |
| Arrangement | Compound | Compound |
| Number of leaflets | 3, 5 sometimes | 3, 5 sometimes |
| Overlapping of lateral leaflets | Free to touching | Free to touching |
| Lateral leaflet: length of stalklet | Very short | Very Short |
| Terminal leaflet | | |
| Length (cm) | 13.5 | 16.5 |
| Width (cm) | 15 | 17.9 |
| Shape | Ovate | Ovate |
| Tip | Acuminate | Acuminate |
| Margin | Double serrate | Double serrate |
| Lateral leaflets (basal pair) | | |
| Length (mm) | 87 | 92 |
| Width | 57 | 54 |
| Overlap | Touching | Touching |
| Orientation | Opposite | Opposite |
| Shape | Ovate | Ovate |
| Tip | Acuminate | Acuminate |
| Base | acute to rounded? | Acute to rounded |
| Margin | Double serrate | Double serrate |
| FLOWERS | | |
| Flowering period | | |
| Primocane | Aug. 15-Sept. 15 | Aug. 1-Sept 1 |
| Floricane | Not harvested | Not harvested |
| Flower diameter (mm) | 18 | 17 |
| Petal | | |
| Length | 6.3 | 6.3 |
| Width | 2.7 | 2.7 |
| FRUIT | | |
| Harvest season | | |
| primocane | 9/15 to frost | 9/1 to frost |
| Fruiting lateral | 8 | 12 |
| length (4th lateral from tip) (cm) | 9 | 7 |
| number of fruit per lateral | 6 | 8 |
| Color | | |
| Immature | 47B | 42B |
| Maturing | 46A | 46A |
| Mature fruit | 59A | 59A |
| Glossiness | Medium | Medium |
| Shape | Conical-ovate | Ovate |
| Dimensions | | |
| Size (g) | | |
| Length (mm) | 21 | 17 |
| Width (mm) | 19.8 | 15 |
| length: width | 1.06 | 1.13 |
| Weight (g/fruit) | | |
| Primocane | 3.5 | 2.9 |
| Soluble solids | 10.8 | 9 |
| Seed weight (g) | 0.002 | 0.008 |
| Number of drupelets/fruit | 70 | 100 |
| Adherence to plug | Medium | Medium |
| Firmness | Medium to Firm | Medium |
| Yield | Low to medium | Medium |

Sensory Evaluation of Nantahala and 4 other red raspberries, Caroline’, “California” (bought off the shelf), and ‘Heritage’ were conducted at the NCSU Dept. Food Science in 2006 (Table 3). ‘Nantahala’ scored as good or better than other cultivars in overall liking, appearance (shape and color), flavor, texture and seediness.

TABLE 3
Sensory Evaluation of ‘Nantahala’ and three other primocane
fruiting red raspberries¹.

| Question | | | | | | | | | |
|---|---|---|---|---|---|---|---|---|---|
| Title | Attribute | Nantahala | | Caroline | | “California” | | Heritage | |
| Overall | Overall | 6.39 | a* | 5.68 | a | 5.77 | a | 5.84 | a |
| Liking | | | | | | | | | |
| Appear- | Red Color | 7.39 | a | 6.56 | bc | 6.53 | bc | 5.89 | c |
| ance | | | | | | | | | |
| Liking | | | | | | | | | |
| Appear- | Shape | 7.23 | a | 6.05 | c | 7.14 | a | 6.26 | bc |
| ance | | | | | | | | | |
| Liking | | | | | | | | | |
| Flavor | Flavor | 6.07 | a | 5.7 | a | 5.61 | a | 5.49 | a |
| Liking | | | | | | | | | |
| Texture | Firmness | 6.16 | a | 4.88 | b | 6.49 | a | 5.81 | a |
| Liking I | | | | | | | | | |
| Texture | Juiciness | 6.67 | ab | 5.93 | b | 6.04 | ab | 6.16 | ab |
| Liking I | | | | | | | | | |
| Seediness/ | Seediness | 2.96 | a | 2.54 | b | 2.49 | b | 2.74 | ab |
| Fuzziness | | | | | | | | | |
| Seediness/ | Fuzziness | 2.18 | b | 2.07 | b | 2.79 | a | 2.32 | b |
| Fuzziness | | | | | | | | | |

*Means in a row followed by different letters are significantly different at the p < 0.05 level
¹Sensory Evaluation Method (NCSU Dept. Food Science)
Consumers scored all products for overall acceptability, red color, shape, flavor, firmness, juiciness, seediness and fuzziness on a 9-point hedonic scale where 9 = like extremely and 1 = dislike extremely.

That which is claimed is:

1. A new and distinct variety of commercial red raspberry (Rubus idaeus Linnaeus) substantially as illustrated and described, characterized by its late season, medium size attractive firm fruit, with moderate and consistent yields.
